(19) United States
(12) Patent Application Publication
(10) Pub. No.: US 2005/0153412 A1
(43) Pub. Date: Jul. 14, 2005

(54) Aldehyde dehydrogenase II

(76) Inventors: Tatsuo Hoshino, Kanagawa-ken (JP); Taro Miyazaki, Kanagawa-ken (JP); Teruhide Sugisawa, Riehen (CH)

Correspondence Address: Stephen M Haracz;Bryan Cave, 1290 Avenue of the Americas, New York, NY, 10104, US

(21) Appl. No.: 10/511,426

(22) PCT Filed: Apr. 14, 2003
(86) PCT No.: PCT/EP03/03862

(30) Foreign Application Priority Data
Apr. 22, 2002 (EP) 02008919.9
Jun. 6, 2002 (EP) 02012584.5

(51) Int. Cl. C12N 9/02; C07H 21/04; C12N 1/21; C12N 15/74

(52) U.S. Cl. 435/189; 435/252.3; 435/471; 435/69.1; 536/23.2

(57) ABSTRACT

The present invention concerns a novel aldehyde dehydrogenase having the following physico-chemical properties: a molecular weight of 100,000±10,000 Da which comprises two homologous subunits or a molecular weight of 150,000±15,000 Da which comprises three homologous subunits, each subunit having a molecular weight of 55,000±2,000 Da; dehydrogenase activity on L-sorbosone, D-Glucosone, D-glucose and D-xylose; utilizes as cofactor pyrroloquinoline quinone; has an optimum pH of from 6.5 to 8.0 for the production of vitamin C and an optimum pH of about 9.0 for the production of 2-keto-L-gulonic acid from L-sorbosone; and is inhibited by Co²⁺, Cu²⁺, Fe³⁺, Ni²⁺, Zn²⁺, and monoiodoacetate, is derived from a microorganism belonging to the genus Gluconobacter.

The present invention concerns a novel enzyme, namely aldehyde dehydrogenase (hereinafter referred to as SNDH II), which is responsible for both of the conversions, from L-sorbosone to L-ascorbic acid (hereinafter referred to as vitamin C) at neutral pH, and from L-sorbosone to 2-keto-L-gulonic acid (hereinafter referred to as 2-KGA) at alkaline pH. The present invention also provides a process for producing said enzyme and a process for producing vitamin C and/or 2-KGA directly from aldoses such as L-sorbosone utilizing said enzyme.

Vitamin C is one of very important and indispensable nutrient factor for human beings. The metabolic pathways to produce vitamin C have been widely studied in various organisms. However, there is no report about purified enzymes relating to the direct conversion of L-sorbosone to vitamin C. Therefore, the enzyme of the present invention is very useful for a novel vitamin C production process substitutive for the current process such as the Reichstein method (Helvetica Chimica Acta 17:311 (1934)).

The present invention provides a purified aldehyde dehydrogenase having the following physico-chemical properties:
a) Molecular weight of 100,000±10,000 Da (consisting of two homologous subunits) or molecular weight of 150,000±15,000 Da (consisting of three homologous subunits), where each subunit has a molecular weight of 55,000±2,000 Da);
b) Substrate specificity: active on aldehyde compounds,
c) Cofactor: pyrroloquinoline quinone (PQQ),
d) Optimum pH of from about 6.5 to about 8.0 (for the production of vitamin C from L-sorbosone) or optimum pH of about 9.0 (for the production of 2-keto-L-gulonic acid from L-sorbosone),
e) Inhibitors: Co²⁺, Cu²⁺, Fe³⁺, Ni²⁺, Zn²⁺, and monoiodoacetate.

In one embodiment, the present invention is related to an aldehyde dehydrogenase with a molecular weight of 100,000±10,000 Da having the physico-chemical properties as described above.

In a further embodiment, the present invention is related to an aldehyde dehydrogenase with a molecular weight of 150,000±15,000 Da having the physico-chemical properties as described above.

The source of the SNDH II of the present invention is not critical. Thus, SNDH II of the present invention can be produced, for example, by isolation from a Gluconobacter or another organism capable of producing the dehydrogenase having the above properties or it can be produced recombinantly or by chemical synthesis.

Another object of the present invention provides a process for producing the SNDH II described above, comprising cultivating a microorganism belonging to the genus Gluconobacter, which is capable of producing the aldehyde dehydrogenase having the above mentioned properties, in an aqueous nutrient medium under aerobic conditions, disrupting the cells of the microorganism, and isolating and purifying the aldehyde dehydrogenase from the cell-free extract of the disrupted cells of the microorganism.

In one aspect of the present invention, the process for producing SNDH II as described above is carried out by cultivating a microorganism belonging to the genus Gluconobacter, which is capable of producing the aldehyde dehydrogenase having the above mentioned properties, wherein the reaction is carried out at a pH of from about 5.5 to about 9.0 and at a temperature of from about 20 to about 50° C., preferably from about 20 to about 40° C., most preferably from about 20 to about 30° C. The SNDH II thus produced is useful for both the production of vitamin C and 2-KGA.

A further object of the present invention provides a process for producing a carboxylic acid and/or its lactone from its corresponding aldose, comprising contacting the aldehyde with the purified SNDH II having the above mentioned properties, or cell-free extract prepared from a microorganism belonging to the genus Gluconobacter which is capable of producing the aldehyde dehydrogenase having the above mentioned properties in the presence of an electron acceptor.

The aldoses as used herein include but are not limited to L-sorbosone, D-glucosone, D-glucose, and D-xylose.

A preferred lactone is vitamin C, a preferred carboxylic acid is 2-KGA and a preferred aldose is L-sorbosone.

In one embodiment, the process for producing a carboxylic acid and/or its lactone from its corresponding aldose comprises contacting the aldehyde with the purified SNDH II having the above mentioned properties or with a cell-free extract prepared from a microorganism belonging to the genus Gluconobacter as defined above, wherein the molecular weight of SNDH II is 100,000±10,000 Da.

In one embodiment, the process for producing a carboxylic acid and/or its lactone from its corresponding aldose comprises contacting the aldehyde with the purified SNDH II having the above mentioned properties or with a cell-free extract prepared from a microorganism belonging to the genus Gluconobacter as defined above, wherein the molecular weight of SNDH II is 150,000±15,000 Da.

In one aspect, the present invention is directed to a process for producing a carboxylic acid and/or its lactone from its corresponding aldose comprising contacting the aldehyde with the purified SNDH II having the above mentioned properties or with a cell-free extract prepared from a microorganism belonging to the genus Gluconobacter, which is capable of producing the aldehyde dehydrogenase having the above mentioned properties, wherein the reaction is carried out at a pH of from about 5.5 to about 9.0 and at a temperature of from about 20 to about 50° C., preferably from about 20 to about 40° C., most preferably from about 20 to about 30° C. In case of the production of vitamin C, the reaction is carried out preferably at a pH of from about 6.5 to about 8.0 and at a temperature of from about 20 to about 40° C. In case of the production of 2-KGA, the reaction is carried out preferably at a pH of about 9.0 and at a temperature of from about 20 to about 30° C.

The invention also features the use of the purified aldehyde dehydrogenase having the above mentioned properties in the process for the production of a carboxylic acid and/or its lactone from its corresponding aldose which comprises contacting the aldehyde with said purified aldehyde dehydrogenase or cell-free extract prepared from a microorganism belonging to the genus Gluconobacter which is capable of producing said aldehyde dehydrogenase in the presence of an electron acceptor.

The physico-chemical properties of the purified sample of SNDH II prepared according to the Examples mentioned hereinafter are as follows:

1) Enzyme Activity

SNDH II of the present invention catalyzes the oxidation of L-sorbosone to vitamin C and/or 2-KGA in the presence of an electron acceptor according to the following reaction equation:
L-Sorbosone+Electron acceptor→Vitamin C and/or 2-KGA+Reduced electron acceptor

The enzyme does not work with oxygen as an electron acceptor. This was affirmed by the failure of the enzyme to convert L-sorbosone to vitamin C and/or 2-KGA using oxygen as a possible electron acceptor. Furthermore, no oxygen consumption was detected in the reaction mixture as detected with a dissolved oxygen probe. In addition NAD and NADP are not suitable electron acceptors. However, other conventional electron acceptors can be utilized in conjunction with the enzyme of this invention. Preferred electron acceptors are phenazine methosulfate (PMS), 2,6-dichlorophenolindophenol (DCIP), ferricyanide and cytochrome c. There is no minimum amount of electron acceptors which must be present for at least some of the aldehyde substrate to be converted to its corresponding acid. However, the amount of substrate which can be oxidized depends on the amount of the particular electron acceptor and its electron accepting characteristics.

The enzyme assay was performed as follows:

a) Assay Determining the Enzyme Activity for the Conversion from L-Sorbosone to Each Product, Vitamin C or 2-KGA

The reaction mixture consisted of 1.0 mM PMS, 25 mM potassium phosphate buffer (pH 7.0), 1.0 μM PQQ, 1.0 mM CaCl₂, 50 mM L-sorbosone and enzyme solution in a final volume of 100 μl water, said reaction mixture was prepared just before the assay. The reaction was carried out at 30° C. for 60 minutes unless otherwise stated. The amount of vitamin C, as the indication for enzyme activity, was measured at a wavelength of 264 nm by a high performance liquid chromatography system (HPLC) which was composed of a UV detector (TOSOH UV8000; TOSOH Co., Kyobashi 3-2-4, Chuo-ku, Tokyo, Japan), a dualpump (TOSOH CCPE; TOSOH Co.), an integrator (Shimadzu C-R6A; Shimadzu Co., Kuwahara-cho 1, Nishinokyo, Chukyo-ku, Kyoto, Japan) and a column (YMC-Pack polyamine II; YMC, Inc., 3233 Burnt Mill Drive Wilimington, N.C. 28403, USA). The amount of produced 2-KGA, as another indication for enzyme activity, was measured by HPLC as described above. One unit of the enzyme activity for each production was defined as the amount of the enzyme which produces 1 mg of vitamin C and 2-KGA, respectively, in the reaction mixture.

b) The Photometrical Assay of SNDH II

The reaction mixture consisted of 0.1 mM DCIP, 1.0 mM PMS, 50 mM potassium phosphate buffer (pH 7.0), 1.0 μM PQQ, 2 to 100 mM substrate (L-sorbosone, D-glucosone, D-glucose, etc.) and enzyme solution in a final volume of 100 μl water, said reaction mixture was prepared just before the assay. The reaction was started at 25° C. with L-sorbosone, and the enzyme activity was measured as the initial reduction rate of DCIP at 600 nm. One unit of the enzyme activity was defined as the amount of the enzyme catalyzing the reduction of 1 μmol DCIP per minute. The extinction coefficient of DCIP at pH 7.0 was taken as 14.2 mM⁻¹. A reference cuvette contained all the above constituents except for L-sorbosone.

The protein concentration was measured with Protein Assay CBB Solution (Nacalai tesque, Inc. Kyoto, Japan).

2) Substrate Specificity

a) The substrate specificity of the enzyme was determined using the same enzyme assay method as described under 1b) above with the exception of using 100 mM potassium phosphate (pH 7.5) or 100 mM Tris-HCl (pH 9.0) as buffer. The relative activity of SNDH II for D-glucosone (2 mM), D-glucose (100 mM), and D-xylose (100 mM) was higher than that for L-sorbosone (2 mM) at both pH 7.5 and 9.0. However, the relative activity for L-sorbose (100 mM), D-sorbitol (100 mM), and L-gulono-γ-lactone (100 mM) was lower than 1% of that for L-sorbosone at both pH 7.5 and 9.0. These results are presented in Table 1A.

TABLE 1A
Substrate specificity of the purified enzyme

Relative activity (%)

| Substrate | pH 7.5 | pH 9.0 |
|---|---|---|
| L-Sorbosone | 100 | 100 |
| D-Glucosone | 483 | 1591 |
| D-Glucose | 1769 | 1519 |
| L-Sorbose | <1 | <1 |
| D-Sorbitol | <1 | <1 |
| D-Xylose | 2123 | 1323 |
| L-Gulono-γ-lactone | <1 | <1 |

b) The deduced products of the oxidation of the substrate indicated in Table 1A are shown in Table 1B below.

TABLE 1B

| Substrate | Product |
|---|---|
| L-Sorbosone | Vitamin C/2-KGA |
| D-Glucosone | D-iso-Ascorbic acid/2-Keto-D-gluconate |
| D-Glucose | D-Gluconate |
| D-Xylose | D-Xylonic acid |

3) Optimum pH

The correlation between the reaction rate of SNDH II and pH values of the reaction mixture was determined by the same assay method as described under 1a) above, with the exception that various pHs and buffers in a concentration of 100 mM were used.

The enzyme showed relatively high activity for the production of vitamin C at a pH of from about 6.5 to about 8.0 and high activity for the production of 2-KGA at a pH of about 9.0.

4) Effect of Temperature

The effect of temperature for the enzyme reaction was tested by the same assay method as described under 1a) above, with the exception that various temperatures were used. In both productions of vitamin C and 2-KGA, the enzyme reaction was carried out stable up to at least 40° C.

5) Effects of Metal Ions and Inhibitors

The effects of metal ions and inhibitors on the L-sorbosone dehydrogenase activity of the enzyme were examined by measuring the activity using the same assay method as described under 1b) above. Each compound solution was stirred into the basal reaction mixture and the reaction was started with the addition of the enzyme. The results are shown in Table 2.

TABLE 2
Effect of inhibitors and metals on
the activity of the purified enzyme

| Compound | Relative activity (%) |
|---|---|
| None | 100.0 |
| EDTA | 97.9 |
| NaN₃ | 98.4 |
| Monoiodoacetate | 34.7 |
| CaCl₂.2H₂O | 94.7 |
| CoCl₂.6H₂O | 60.8 |
| CuSO₄ | <1 |
| Fe2(SO₄)₃.xH₂O | 73.2 |
| NiSO₄.6H₂O | 82.9 |
| TiCl₄ | 95.9 |
| ZnCl₂ | 64.9 |
| MgCl₂ | 88.5 |

Each compound was added to the reaction mixture at a concentration of 1.0 mM, with the exception that the concentrations of EDTA, NaN₃ and monoiodoacetate were 5.0 mM.

As shown in Table 2, Co²⁺, Cu²⁺, Fe³⁺, Ni²⁺, and Zn²⁺ inhibited the enzyme activity. The addition of 5.0 mM monoiodoacetate strongly inhibited the enzyme activity.

6) Molecular Weight

The molecular weight of the enzyme was measured with a size exclusion gel column (TSK-gel G3000 SWXL; TOSOH Co., Akasaka 1-7-7, Minato-ku, Tokyo, Japan). The enzyme showed two peaks corresponding to the apparent molecular weight of about 100,000±10,000 Da and about 150,000±15,000 Da on the chromatography. By analyzing this enzyme on 10% SDS-polyacrylamide gel electrophoresis stained with CBB, it was shown that the purified enzyme consisted of two to three homologous subunits each having a molecular weight of about 55,000±2,000 Da. Both the dimeric and trimeric forms of the enzyme are active.

7) Prosthetic Group

The purified SNDH II (0.1 mg) in 50 μl of 100 mM NaH₂PO₄—HCl (pH about 1.0) was added by an equal volume of methanol and mixed well. The sample was centrifuged to remove a precipitate. The resulting supernatant was used for analysis of the prosthetic group. The absorption spectrum of the extract was almost identical with an authentic sample of PQQ (Mitsubishi Gas Chemical, Japan). Its absorbance peaks were found at 251 and 348 nm. Furthermore, by HPLC analysis using a reverse phase column (YMC-Pack Pro C18 AS-312; YMC Co., Ltd) at a wavelength of 313 nm, the extract of SNDH II with methanol showed the same retention time as that of the authentic PQQ.

The detection of heme c of the purified enzyme was attempted by the reduced-minus-oxidized difference spectrum taken by a UV-VIS recording spectrophotometer (Shimadzu UV-2200; Shimadzu Co.). The enzyme was suspended in 50 mM potassium phosphate buffer (pH 7.0) at a concentration of 50 μg/ml and the enzyme of dithionite-reduced form and ammonium persulfate-oxidized form were prepared to measure the difference spectrum. However, the spectrum obtained did not show apparent peaks at a wavelength of between 450 and 650 nm.

These results strongly suggest that the enzyme has PQQ, but has no heme c as prosthetic group.

8) Effect of Substrate Concentration

The velocity of the oxidizing reaction with various concentrations of L-sorbosone from 1 mM to 8 mM was measured to determine the Km value for L-sorbosone. The Michaelis constants were calculated to be 14.7 mM and 20.0 mM at pHs of 7.5 and 9.0, respectively, from the Lineweaver-Burk plot based on the reaction velocity when DCIP was used as the electron acceptor for the reaction.

9) Purification Procedure

The purification of the enzyme is effected by any combination of known purification methods, such as ion exchange column chromatography, hydrophobic column chromatography, salting out and dialysis.

The enzyme provided by the present invention can be prepared by cultivating an appropriate microorganism in an aqueous nutrient medium under aerobic conditions, disrupting the cells of the microorganism and isolating and purifying the dehydrogenase from the cell-free extract of the disrupted cells of the microorganism.

The microorganisms used for the process of the present invention are microorganisms belonging to the genus Gluconobacter which are capable of producing the dehydrogenase as defined herein before.

A preferred strain is Gluconobacter oxydans. The strain most preferably used in the present invention is Gluconobacter oxydans DSM 4025, which was deposited at the Deutsche Sammlung von Mikroorganismen in Göttingen (Germany), based on the stipulations of the Budapest Treaty, under DSM No. 4025 on Mar. 17, 1987. The depositor was The Oriental Scientific Instruments Import and Export Corporation for Institute of Microbiology, Academia Sinica, 52 San-Li-He Rd., Beijing, Peoples Republic of China. The effective depositor was said Institute, of which the full address is The Institute of Microbiology, Academy of Sciences of China, Haidian, Zhongguancun, Beijing 100080, People's Republic of China.

Moreover, a subculture of the strain has also been deposited at the National Institute of Advanced Industrial Science and Technology (AIST), Japan, also based on the stipulations of the Budapest Treaty, under the deposit No. Gluconobacter oxydans DSM No. 4025 (FERM BP-3812) on Mar. 30, 1992. The depositor is Nippon Roche K.K., 6-1, Shiba 2-chome, Minato-ku, Tokyo, Japan. This subculture is also most preferably used in the present invention.

Thus, it is an object of the present invention to provide an aldehyde dehydrogenase as defined herein before which is derived from Gluconobacter oxydans having the identifying characteristics of the strain Gluconobacter oxydans DSM No. 4025 (FERM BP-3812), a subculture or mutant thereof.

Mutants of G. oxydans DSM 4025 (FERM BP-3812) or a microorganism belonging to the genus Gluconobacter and having identifying characteristics of G. oxydans DSM 4025 (FERM BP-3812) may be obtained by treating the cells by means of, for instance, ultraviolet or X-ray irradiation, or a chemical mutagen such as nitrogen mustard or N-methyl-n′-nitro-N-nitrosoguanidine.

Any type of microorganism may be used, for instance, resting cells, acetone treated cells, lyophilized cells, immobilized cells and the like to act directly on the substrate. Any means per se known as a method in connection with the incubation technique for micro-organisms may be adopted through the use of aeration and agitated submerged fermenters is particularly preferred. The preferred cell concentration range for carrying out the reaction is from about 0.01 g of wet cell weight per ml to 0.7 g of wet cell per ml, preferably from 0.03 g of wet cell per ml to 0.5 g of wet cell per ml.

The microorganism “Gluconobacter oxydans” also includes synonyms or basonyms of such species having the same physico-chemical properties, as defined by the International Code of Nomenclature of Prokaryotes.

The characteristics of G. oxydans DSM No. 4025 (FERM BP-3812) are as follows:
a) production of 2-KGA from sorbose,
b) ethanol is oxidized to acetic acid,
c) D-glucose is oxidized to D-gluconic acid and 2-keto-D-gluconic acid,
d) ketogenesis of polyalcohols,
e) pellicle and ring growth in mannitol broth (24 h cultivation) at pH 4 and 5, and pellide growth in glucose broth at pH 4.5,
f) glycerol is not substantially oxidized to dihydroxyacetone,
g) production of 2-keto-D-glucaric acid from sorbitol and glucaric acid but not from glucose, fructose, gluconic acid, mannitol or 2-keto-D-gluconic acid,
h) polymorphic, apparently no flagella,
i) brown pigment is produced from fructose,
j) good growth when co-cultured in the presence of Bacillus megaterium or a cell extract thereof,
k) streptomycin sensitive.

The microorganism may be cultured in an aqueous medium supplemented with appropriate nutrients under aerobic conditions. The cultivation may be conducted at a pH of from about 4.0 to about 9.0, preferably from about 6.0 to about 8.0. The cultivation period varies depending on the pH, temperature and nutrient medium to be used, and is preferably about 1 to 5 days. The preferred temperature for carrying out the cultivation is from about 13° C. to about 36° C., more preferably from about 18° C. to about 33° C. A temperature of up to about 50° C. might be also suitable for the cultivation of the microorganism.

It is usually required that the culture medium contains such nutrients as assimilable carbon sources, for example glycerol, D-mannitol, D-sorbitol, erythritol, ribitol, xylitol, arabitol, inositol, dulcitol, D-ribose, D-fructose, D-glucose, and sucrose, preferably D-sorbitol, D-mannitol and glycerol; and digestible nitrogen sources such as organic substances, for example, peptone, yeast extract, baker's yeast, urea, amino acids, and corn steep liquor. Various inorganic substances may also be used as nitrogen sources, for example nitrates and ammonium salts. Furthermore, the culture medium usually contains inorganic salts, for example magnesium sulfate, potassium phosphate and calcium carbonate.

An embodiment for the isolation and purification of SNDH II from the microorganism after the cultivation is briefly described hereinafter:
(1) Cells are harvested from the liquid culture broth by centrifugation or filtration.
(2) The harvested cells are washed with water, physiological saline or a buffer solution having an appropriate pH.
(3) The washed cells are suspended in the buffer solution and disrupted by means of a homogenizer, sonicator or French press or by treatment with lysozyme and the like to give a solution of disrupted cells.
(4) SNDH II is isolated and purified from the cell-free extract of disrupted cells, preferably from the soluble fraction of the microorganism.

A cell free extract can be obtained from the disrupted cells by any conventional technique, including but not limited to centrifugation.

SNDH II provided by the present invention is useful as a catalyst for the production of vitamin C and/or 2-KGA from L-sorbosone. The reaction can be conducted at a pH of from about 5.5 to about 9.0 for both of vitamin C production and 2-KGA production in the presence of an electron acceptor, for example DCIP, PMS and the like in a solvent such as phosphate buffer, Tris-buffer and the like. For vitamin C production, the best results are usually achieved if the pH is set at about 6.5 to about 8.0 and the temperature is set at about 20 to about 40° C. For 2-KGA production, the best results are usually achieved if the pH is set at about 9.0 and the temperature is set at about 20 to about 30° C.

The concentration of L-sorbosone in a reaction mixture can vary depending upon other reaction conditions but, in general, is about 0.5 to about 50 g/l, most preferably from about 1 to about 30 g/l.

In the reaction, SNDH II may also be used in an immobilized state with an appropriate carrier. Any means of immobilizing enzymes generally known in the art may be used. For instance, the enzyme may be bound directly to a membrane, granules or the like of a resin having one or more functional groups, or it may be bound to the resin through bridging compounds having one or more functional groups, for example glutaraldehyde.

In addition to the above, the cultured cells are also useful for the production of carboxylic acids and/or its lactones from their corresponding aldoses, especially for the production of 2-KGA and/or vitamin C from L-sorbosone. The production of other carboxylic acids and/or its lactones from their corresponding aldoses is carried out under the same conditions, including substrate concentration, as the conversion of L-sorbosone to 2-KGA and/or vitamin C as described above.

The following Examples further illustrate the present invention.

### EXAMPLE 1

### Preparation of SNDH II

All the operations were performed at 8° C., and the buffer was 0.05 M potassium phosphate (pH 7.0) unless otherwise stated.

(1) Cultivation of Gluconobacter oxydans DSM No. 4025 (FERM BP-3812)

Gluconobacter oxydans DSM No. 4025 (FERM BP-3812) was grown on an agar plate containing 5.0% D-mannitol, 0.25% MgSO₄.7H₂O, 1.75% corn steep liquor, 5.0% baker's yeast, 0.5% urea, 0.5% CaCO₃ and 2.0% agar at 27° C. for 4 days. One loopful of the cells was inoculated into 50 ml of a seed culture medium containing 2% L-sorbose, 0.2% yeast extract, 0.05% glycerol, 0.25% MgSO₄.7H₂O, 1.75% corn steep liquor, 0.5% urea and 1.5% CaCO₃ in a 500 ml Erlenmeyer flask, and cultivated at 30° C. with 180 rpm for one day on a rotary shaker. The seed culture thus prepared was used for inoculating 2 liters of medium, which contained 8.0% L-sorbose, 0.05% glycerol, 0.25% MgSO₄.7H₂O, 3.0% corn steep liquor, 0.4% yeast extract and 0.15% antifoam, in a 3-1 jar fermentor. The fermentation parameters were 800 rpm for the agitation speed and 0.5 vvm (volume of air/volume of medium/minute) for aeration at a temperature of 30° C. The pH was maintained at 7.0 with sodium hydroxide during the fermentation. After 48 hours of cultivation, 6 liters of the cultivated broth containing the cells of Gluconobacter oxydans DSM No. 4025 (FERM BP-3812) by using the three sets of fermentors were harvested by continuous centrifugation. The pellets containing the cells were recovered and suspended in an appropriate volume of saline. After the suspension was centrifuged at 2,500 rpm (1,000×g), the supernatant containing the slightly reddish cells was recovered to remove the insoluble materials derived from corn steep liquor and yeast extract which were ingredients for the medium. The supernatant was then centrifuged at 8,000 rpm (10,000×g) to obtain the cell pellet. As a result, 38.4 g of the wet weight of cells of Gluconobacter oxydans DSM No. 4025 (FERM BP-3812) was obtained from 6 liters of the broth.

(2) Preparation of Cytosol Fraction

A portion (19.2 g) of the cell paste was suspended with 100 ml of the buffer and passed through a French pressure cell press. After centrifugation to remove intact cells, the supernatant was designated as the cell-free extract, and the cell-free extract was centrifuged at 100,000×g for 60 minutes. The resultant supernatant (112 ml) was designated as the soluble fraction of Gluconobacter oxydans DSM No. 4025 (FERM BP-3812). After this fraction was dialyzed against the buffer, 112 ml of the dialyzed fraction having the specific activity for producing vitamin C from L-sorbosone of 0.172 unit/mg protein were used for the next purification step.

(3) Diethylaminoethyl (DEAE)-Cellulose Column Chromatography

The dialysate (112 ml) was put on a column of DEAE-cellulose (Whatman DE-52, 3×50 cm; Whatman BioSystems Ltd., Springfield MIII, James Whatman Way, Maidstone, Kent, U.K.) equilibrated with the buffer and washed with the buffer to elute minor proteins. Then a linear gradient elution with NaCl from 0.28 to 0.58 M in the buffer was carried out. Major enzyme activity was eluted at 0.36 M NaCl. The active fractions (97.5 ml) were collected.

(4) DEAE-Sepharose Column Chromatography

A portion (97 ml) of the dialyzed active fraction from the previous step was put on a column of DEAE-sepharose CL-6B (Pharmacia, 3.0 by 25 cm) equilibrated with the buffer. After the column was washed with the buffer containing 0.3 M NaCl, a linear gradient of NaCl from 0.3 to 0.45 M was added to the buffer. The active fractions were eluted at NaCl concentrations ranging from 0.44 to 0.47 M. The active fractions (40 ml) were collected and dialyzed against the buffer.

(5) Q-Sepharose Column Chromatography (1^{st} Step)

The dialyzed active fraction (40 ml) was put on a column of Q-sepharose (Pharmacia, 1.5 by 25 cm) equilibrated with the buffer. After the column was washed with the buffer containing 0.3 M NaCl, a linear gradient of NaCl from 0.3 to 0.5 M was added to the buffer. The active fractions were eluted at NaCl concentrations ranging from 0.44 to 0.46 M.

(6) Q-Sepharose Column Chromatography (2^{nd} Step)

The pooled active fractions (17 ml) from the previous step were dialyzed against the buffer. The dialyzed sample (17 ml) was put on a column of Q-sepharose (Pharmacia, 1.5 by 25 cm) equilibrated with the buffer. After the column was washed with the buffer containing 0.33 M NaCl, a linear gradient of NaCl from 0.33 to 0.48 M was added to the buffer. The active fractions were eluted at NaCl concentrations ranging from 0.45 to 0.48 M.

(7) Hydrophobic Column Chromatography

The active fraction from the previous step was filtrated by an ultrafiltrator (Centriprep-10) to desalt and concentrate. A portion (750 μl) of the desalted and concentrated sample (780 μl) was added to the equal volume (750 μl) of the buffer containing 3 M ammonium sulfate (the final concentration: 1.5 M). After centrifugation (15,000×g) of the sample, the supernatant was put on a hydrophobic column RESOURCE ISO (Pharmacia, bed volume: 1.0 ml) equilibrated with the buffer containing 1.5 M ammonium sulfate. After the column was washed with the buffer containing 1.5 M ammonium sulfate, the proteins were eluted with the buffer containing a linear gradient of ammonium sulfate from 1.5 to 0.75 M. The activities corresponding to SNDH II were eluted at ammonium sulfate concentrations ranging from 1.04 to 1.00 M. The active fractions were dialyzed against the buffer using dialysis cups (Dialysis-cup MWCO 8000, Daiichi pure chemicals, Nihonbashi 3-13-5, Chuo-ku, Tokyo, Japan). Afterward, the fractions were gathered and stored at −20° C.

A summary of the purification steps of the enzyme is given in Table 3.

TABLE 3
Purification of the aldehyde dehydrogenase
from Gluconobacteroxydans DSM No.
4025 (FERM BP-3812)

| | Total | Total | Specific activity |
|---|---|---|---|
| | activity | protein | (units*/mg |
| Step | (units) | (mg) | protein) |
| Soluble fraction | 151.4 | 879.3 | 0.172 |
| DEAE-Cellulose DE52 | 173.0 | 37.73 | 4.584 |
| DEAE-Sepharose CL-6B | 45.07 | 10.63 | 4.242 |
| Q-Sepharose (1^{st} step) | 23.65 | 1.462 | 16.17 |
| Q-Sepharose (2^{nd} step) | 13.70 | 0.527 | 26.03 |
| RESOURCE-ISO | 4.84 | 0.099 | 48.90 |

One unit* of the enzyme was defined as the amount of enzyme which produces 1 mg of vitamin C per hour in the reaction mixture described in 1a).
(8) Purity of the Isolated Enzyme

The purified enzyme (0.039 mg/ml) with a specific activity of 48.9 units per mg protein for vitamin C production and a specific activity of 12.3 units per mg protein for 2-KGA production was used for the following analysis:

The molecular weight of the native enzyme was estimated by high performance liquid chromatography using a size exclusion gel column (TSK gel G3000 SWXL column, 7.8×300 mm) equilibrated with 0.1 M potassium phosphate buffer (pH 7.0) containing 0.3 M NaCl at 280 nm and a flow rate of 1.5 ml per minute. Cyanocobalamin (1.35 kDa), myoglobin (17 kDa), ovalbumin (44 kDa), γ-globulin (158 kDa) and thyroglobulin (670 kDa) were used as molecular weight standards. The purified enzyme showed two peaks having the molecular weight of 100,000±10,000 Da and 150,000±15,000 Da, respectively.

According to sodium dodecyl sulfate polyacrylamide gel electrophoresis (SDS-PAGE), the enzyme showed a subunit with a molecular weight of 55,000±2,000 Da. Therefore, the purified enzyme was estimated to consist of two or three homologous subunits.

(9) Identification of the Reaction Product

The reaction mixture containing the purified enzyme (0.39 μg), L-sorbosone (50 mM), PMS (1 mM), CaCl₂ (1 mM) and PQQ (1 μM) was incubated in 100 μl of the buffer for 1 hour at 30° C. The reaction products were analyzed on thin layer chromatography (Silica gel 60F²⁵⁴, MERCK, 64271 Darmstadt, Germany) and HPLC. Two kinds of products, vitamin C and 2-KGA, were obtained from the enzyme reaction. For vitamin C, the sample was assayed by an amino-column (YMC-Pack Polyamine-II, YMC, Inc.) on a HPLC system. For 2-KGA, the sample was assayed by a C-18 column (YMC-Pack Pro C18, YMC, Inc.) on a HPLC system.

### EXAMPLE 2

### Effect of pH on the Production of Vitamin C or 2-KGA from L-Sorbosone by SNDH II

The effect of pH for the enzyme reaction was tested. The reaction mixture containing the purified enzyme (273 ng), L-sorbosone (50 mM), PMS (1 mM), CaCl₂ (1 mM) and PQQ (1 μM) in 100 μl of the buffer (100 mM) was incubated for 1 hour at 30° C. The reaction products were analyzed by HPLC. The result is shown in Table 4.

TABLE 4
Effect of pH on the production of vitamin
C or 2-KGA from L-sorbosone by SNDH II

| | | Vitamin C produced | 2-KGA produced |
|---|---|---|---|
| Buffer | pH | (mg/l) | (mg/l) |
| Citrate-NaOH | 4.50 | 0.0 | 0.0 |
| Citrate-NaOH | 5.50 | 3.8 | 27.7 |
| Citrate-NaOH | 6.50 | 64.7 | 21.2 |
| Potassium phosphate | 6.76 | 7.8 | not done |
| Potassium phosphate | 7.15 | 64.4 | not done |
| Potassium phosphate | 7.55 | 76.3 | 1.0 |
| Potassium phosphate | 7.97 | 49.6 | 19.5 |
| Tris-HCl | 7.86 | 70.7 | 117.8 |
| Tris-HCl | 8.34 | 18.5 | 124.0 |
| Tris-HCl | 8.83 | 7.2 | 170.7 |

### EXAMPLE 3

### Effect of Temperature on the Production of Vitamin C or 2-KGA from L-Sorbosone by SNDH II

The effect of temperature on the enzyme activity was tested. The reaction mixture containing the purified enzyme (390 ng), L-sorbosone (50 mM), PMS (1 mM), CaCl₂ (1 mM) and PQQ (1 μM) in 100 μl of 25 mM potassium phosphate buffer (pH 7.0) was incubated for 1 hour at various temperatures (20-60° C.). The reaction products were analyzed by HPLC. The result is shown in Table 5.

TABLE 5
Effect of temperature on the production of vitamin
C or 2-KGA from L-sorbosone by SNDH II

| Temperature | Vitamin C produced | 2-KGA produced |
|---|---|---|
| (° C.) | (mg/l) | (mg/l) |
| 20 | 187.4 | 50.6 |
| 25 | 218.8 | 53.5 |
| 30 | 190.7 | 48.1 |
| 35 | 196.4 | 40.3 |
| 40 | 176.7 | 37.2 |
| 50 | 138.0 | 32.8 |
| 60 | 47.3 | 4.3 |

1. A purified aldehyde dehydrogenase having the following physico-chemical properties:
a) Molecular weight of 100,000±10,000 Da (consisting of two homologous subunits) or molecular weight of 150,000±15,000 Da (consisting of three homologous subunits), where each subunit has a molecular weight of 55,000±2,000 Da);
b) Substrate specificity: active on L-sorbosone, D-glucosone, D-glucose, D-xylose;
c) Cofactor: pyrroloquinoline quinone (PQQ),
d) Optimum pH of from about 6.5 to about 8.0 (for the production of vitamin C from L-sorbosone) or optimum pH of about 9.0 (for the production of 2-keto-L-gulonic acid from L-sorbosone),
e) Inhibitors: Co²⁺, Cu²⁺, Fe³⁺, Ni²⁺, Zn²⁺, and monoiodoacetate.

2. The aldehyde dehydrogenase according to claim 1, which is derived from a microorganism belonging to the genus Gluconobacter which is capable of producing said aldehyde dehydrogenase.

3. The aldehyde dehydrogenase according to claim 2, wherein the microorganism is Gluconobacter oxydans having the identifying characteristics of the strain Gluconobacter oxydans DSM No. 4025 (FERM BP-3812), a subculture or mutant thereof.

4. The aldehyde dehydrogenase according to claim 3, wherein the microorganism is Gluconobacter oxydans DSM No. 4025 (FERM BP-3812), a subculture or mutant thereof.

5. A process for producing an aldehyde dehydrogenase having the following physico-chemical properties:
a) Molecular weight of 100,000±10,000 Da (consisting of two homologous subunits) or molecular weight of 150,000±15,000 Da (consisting of three homologous subunits), where each subunit has a molecular weight of 55,000±2,000 Da);
b) Substrate specificity: active on aldehyde compounds,
c) Cofactor: pyrroloquinoline quinone (PQQ),
d) Optimum pH of from about 6.5 to about 8.0 (for the production of vitamin C from L-sorbosone) or optimum pH of about 9.0 (for the production of 2-keto-L-gulonic acid from L-sorbosone),
e) Inhibitors: Co²⁺, Cu²⁺, Fe³⁺, Ni²⁺, Zn²⁺, and monoiodoacetate, which comprises cultivating a microorganism belonging to the genus Gluconobacter, which is capable of producing the aldehyde dehydrogenase having the above properties, in an aqueous nutrient medium under aerobic conditions, disrupting the cells of the microorganism, and isolating and purifying the aldehyde dehydrogenase from the cell-free extract of the disrupted cells of the microorganism.

6. The process according to claim 5, wherein the reaction is carried out at a pH of from about 5.5 to 9.0 and at a temperature of from about 20 to about 50° C.

7. A process for producing a carboxylic acid and/or its lactone from its corresponding aldose which comprises contacting the aldehyde with the purified aldehyde dehydrogenase having the following physico-chemical properties:
a) Molecular weight of 100,000±10,000 Da (consisting of two homologous subunits) or molecular weight of 150,000±15,000 Da (consisting of three homologous subunits), where each subunit has a molecular weight of 55,000±2,000 Da);
b) Substrate specificity: active on aldehyde compounds,
c) Cofactor: pyrroloquinoline quinone (PQQ),
d) Optimum pH of from about 6.5 to about 8.0 (for the production of vitamin C from L-sorbosone) or optimum pH of about 9.0 (for the production of 2-keto-L-gulonic acid from L-sorbosone),
e) Inhibitors: Co, Cu²⁺, Fe³⁺, Ni²⁺, Zn²⁺, and monoiodoacetate, or cell-free extract prepared from a microorganism belonging to the genus Gluconobacter which is capable of producing the aldehyde dehydrogenase having the above properties in the presence of an electron acceptor.

8. The process according to claim 5, wherein the microorganism is Gluconobacter oxydans having the identifying characteristics of the strain Gluconobacter oxydans DSM No. 4025 (FERM BP-3812), a subculture or mutant thereof.

9. The process according to claim 8, wherein the microorganism is Gluconobacter oxydans DSM No. 4025 (FERM BP-3812), a subculture or mutant thereof.

10. The process of claim 7, wherein the lactone is vitamin C, the carboxylic acid is 2-keto-L-gulonic acid and the aldose is L-sorbosone.

11. The process according to claim 7, wherein the reaction is carried out at a pH of from about 5.5 to about 9.0 and at a temperature of from about 20 to about 50° C. for the production of vitamin C and 2-keto-L-gulonic acid, respectively.

12. The process according to claim 7, wherein the reaction is carried out at a pH of from about 6.5 to about 8.0 and a temperature of from about 20 to about 40° C. for the production of vitamin C, and at a pH of about 9.0 and a temperature of from about 20 to about 30° C. for the production of 2-keto-L-gulonic acid.

13. A process for the production of a carboxylic acid and/or its lactone from its corresponding aldose which comprises contacting the aldehyde with the purified aldehyde dehydrogenase of claim 1 or cell-free extract prepared from a microorganism belonging to the genus Gluconobacter which is capable of producing said aldehyde dehydrogenase in the presence of an electron acceptor.

14. The process according to claim 6, wherein the microorganism is Gluconobacter oxydans having the identifying characteristics of the strain Gluconobacter oxydans DSM No. 4025 (FERM BP-3812), a subculture or mutant thereof.

15. The process according to claim 7, wherein the microorganism is Gluconobacter oxydans having the identifying characteristics of the strain Gluconobacter oxydans DSM No. 4025 (FERM BP-3812), a subculture or mutant thereof.

16. The process according to claim 8, wherein the reaction is carried out at a pH of from about 5.5 to about 9.0 and at a temperature of from about 20 to about 50° C. for the production of vitamin C and 2-keto-L-gulonic acid, respectively.

17. The process according to claim 9, wherein the reaction is carried out at a pH of from about 5.5 to about 9.0 and at a temperature of from about 20 to about 50° C. for the production of vitamin C and 2-keto-L-gulonic acid, respectively.

18. The process according to claim 10, wherein the reaction is carried out at a pH of from about 5.5 to about 9.0 and at a temperature of from about 20 to about 50° C. for the production of vitamin C and 2-keto-L-gulonic acid, respectively.

19. The process according to claim 8, wherein the reaction is carried out at a pH of from about 6.5 to about 8.0 and a temperature of from about 20 to about 40° C. for the production of vitamin C, and at a pH of about 9.0 and a temperature of from about 20 to about 30° C. for the production of 2-keto-L-gulonic acid.

20. The process according to claim 9, wherein the reaction is carried out at a pH of from about 6.5 to about 8.0 and a temperature of from about 20 to about 40° C. for the production of vitamin C, and at a pH of about 9.0 and a temperature of from about 20 to about 30° C. for the production of 2-keto-L-gulonic acid.

21. The process according to claim 10, wherein the reaction is carried out at a pH of from about 6.5 to about 8.0 and a temperature of from about 20 to about 40° C. for the production of vitamin C, and at a pH of about 9.0 and a temperature of from about 20 to about 30° C. for the production of 2-keto-L-gulonic acid.

22. The process according to claim 11, wherein the reaction is carried out at a pH of from about 6.5 to about 8.0 and a temperature of from about 20 to about 40° C. for the production of vitamin C, and at a pH of about 9.0 and a temperature of from about 20 to about 30° C. for the production of 2-keto-L-gulonic acid.
